(19) United States
(12) Patent Application Publication
(10) Pub. No.: US 2015/0265856 A1
(43) Pub. Date: Sep. 24, 2015

(54) SYSTEMS, METHODS AND DEVICES FOR PRECISION HIGH-INTENSITY FOCUSED ULTRASOUND

(71) Applicant: LaZure Scientific, Inc., Issaquah, WA (US)

(72) Inventor: Charles E. Hill, Issaquah, WA (US)

(21) Appl. No.: 14/416,552

(22) PCT Filed: Jul. 23, 2013
(86) PCT No.: PCT/US13/51591

§ 371 Date: Jan. 22, 2015

Related U.S. Application Data
(60) Provisional application No. 61/674,668, filed on Jul. 23, 2012.

(51) Int. Cl. A61N 7/02

(52) U.S. Cl. CPC A61N 7/02

(57) ABSTRACT

Methods, systems, and treatment probes for delivering heating energy such as acoustic waves to a target tissue volume inside of a patient for medically treating the target tissue volume are disclosed. A method includes inserting a treatment probe into the patient through an exposed skin of the patient, the treatment probe including heating energy dispensing element. The method further includes applying heating energy to the target tissue volume via the dispensing element, the heating energy being applied so as to medically treat the target tissue volume. The method also includes monitoring an amount of energy absorbed by the target tissue as a result of applying the energy, and adjusting the heating energy being applied to the target tissue based on the amount of energy absorbed by the target tissue.

### CROSS-REFERENCE

This application claims the benefit of U.S. Provisional Application No. 61/674,668, filed Jul. 23, 2012, which application is incorporated herein by reference.

### BACKGROUND OF THE INVENTION

High intensity focused ultrasound (HIFU) has been used in medical applications for a number of years, where HIFU transducers are arranged outside of a patient's body and focus ultrasound waves to a target location inside of the body. The primary effect of high acoustic intensities in tissue is heat generation due to the acoustic energy absorption. In most HIFU applications, the heat generated rapidly raises the temperature in the target tissue to 60 degrees Celcius or higher causing coagulation necrosis within a few seconds.

Other effects of applying high acoustic intensities include a cavitation effect as the acoustic field causes the movement of gas-filled bubbles in a liquid medium. This cavitation occurs due to the expansion and compression of tissue as the ultrasound field propagates through it. If inertial cavitation occurs there is the possibility of a violent collapse and destruction of the bubble. If this collapse occurs near a cell membrane, mechanical damage to the cell membrane is possible due to high velocity liquid jets impacting the cell wall as the bubble collapses. Microstreaming may also occur, in which high shear forces close to the oscillating bubble cause cell membrane disruption. Further, radiation forces may also occur when a wave is either absorbed or reflected, producing radiation pressure, and cell death may be caused by apoptosis following HIFU treatment.

Numerous problems arise with current HIFU delivery methods. Some problems include acoustic shadowing, reverberation, and refraction. Such problems result in extreme difficulty in treating areas that are deep in the tissue and/or are impacted by bone structures (such as treating liver tissue close to a rib). Another problem is that gas in the body (e.g., the bowel) cannot be penetrated by HIFU and the sound waves are reflected back toward the transducer, possibly resulting in non-target tissue being damaged via burns to the tissue that lies between the transducer and the target. Yet another problem is that current systems estimate the amount of energy absorbed by making the assumption that the attenuation of the sound waves in the soft tissues between the transducer and the target location is linear. However, this is rarely the case as fibrotic, fatty, and vascularized tissues attenuate the sound energy differently, and since there is a heat sink effect associated with vascularity. Further, one potential complication that has not yet been substantiated is the possibility of the dissemination of malignant cells from the shear forces generated by the procedure.

### SUMMARY OF THE INVENTION

Embodiments of the present invention overcome one or more of the problems associated with prior art HIFU systems. According to some embodiments, a method of delivering acoustic waves to a target tissue volume inside of a patient for medically treating the target tissue volume is disclosed. The method includes inserting a treatment probe into the patient through an exposed skin of the patient, the treatment probe including an acoustic wave dispensing element. The method also includes applying acoustic waves to the target tissue volume via the acoustic wave dispensing element, the acoustic waves being applied so as to medically treat the target tissue volume. The method further includes monitoring an amount of energy absorbed by the target tissue as a result of applying the acoustic waves, and adjusting the acoustic waves being applied to the target tissue based on the amount of energy absorbed by the target tissue.

According to other embodiments, a system for delivering acoustic waves to a target tissue volume inside of a patient for medically treating the target tissue volume is disclosed. The system includes a treatment probe for treating the target tissue volume, the probe being insertable through an exposed skin of the patient and including an acoustic wave dispensing element operable to output acoustic waves for medically treating the target tissue volume. The system further includes a monitor operable to monitor an amount of energy absorbed by the target tissue as a result of applying the acoustic waves. The system also includes a controller coupled to the wave dispensing element and the monitor, the controller operable to control the acoustic waves output by the wave dispensing element based on the amount of energy absorbed by the target tissue.

According to yet other embodiments, a treatment probe for delivering acoustic waves to a target tissue volume inside of a patient for medically treating the target tissue volume is disclosed. The treatment probe includes a housing having one end configured to pierce through exposed skin of the patient, an acoustic wave dispensing element coupled to the housing and operable to output acoustic waves, and a communication element coupled to the acoustic wave dispensing element operable to communicate signals for controlling the acoustic waves.

For a fuller understanding of the nature and advantages of the present invention, reference should be made to the ensuing detailed description and accompanying drawings. Other aspects, objects and advantages of the invention will be apparent from the drawings and detailed description that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a block diagram of a simplified system for selectively applying acoustic waves to target volumes in accordance with an embodiment.

FIG. 2A illustrates a treatment probe applying acoustic waves to a target volume in accordance with an embodiment.

FIG. 2B illustrates a treatment probe applying acoustic waves to a target volume in accordance with an embodiment.

FIG. 2C illustrates a treatment probe applying acoustic waves to a target volume in accordance with an embodiment.

FIG. 2D illustrates a treatment probe applying acoustic waves to a target volume in accordance with an embodiment.

FIG. 2E illustrates a treatment probe applying acoustic waves to a target volume in accordance with an embodiment.

FIG. 2F illustrates a treatment probe applying acoustic waves to a target volume in accordance with an embodiment.

FIG. 2G illustrates a treatment probe applying acoustic waves to a target volume in accordance with an embodiment.

FIG. 2H illustrates an array of treatment probes applying heating energy to a target volume in accordance with an embodiment.

FIG. 2I illustrates a cross-section front view of a tissue volume having an array of treatment probes as in FIG. 2H in accordance with an embodiment.

FIG. 3A is a profile view of a treatment probe according to a first embodiment.

FIG. 3B is a cross-sectional view of the treatment probe of FIG. 3A.

FIG. 4A is a profile view of a treatment probe according to a second embodiment.

FIG. 4B is a cross-sectional view of the treatment probe of FIG. 4A.

FIG. 5A is a cross-sectional view of a treatment probe including an acoustic transducer according to a first embodiment.

FIG. 5B is a cross-sectional view of a treatment probe including an acoustic transducer according to a second embodiment.

FIG. 5C is a cross-sectional view of a treatment probe including an acoustic transducer according to a third embodiment.

FIG. 6A is a cross-sectional view of a treatment probe including an acoustic lens according to a first embodiment.

FIG. 6B is a cross-sectional view of a treatment probe including an acoustic lens according to a second embodiment.

FIG. 6C is a cross-sectional view of a treatment probe including an acoustic lens according to a third embodiment.

FIG. 7A illustrates a treatment probe externally applying energy to a target volume located in an object while a temperature probe is disposed in the target volume.

FIG. 7B illustrates a treatment probe internally applying energy to a target volume located in an object while a temperature probe is disposed in the target volume.

FIG. 8 is a flowchart depicting example operations of a method for treating a target volume using acoustic waves according to a first embodiment.

FIG. 9 is a flowchart depicting example operations of a method for treating a target volume using acoustic waves according to a second embodiment.

### DETAILED DESCRIPTION OF THE INVENTION

Many of the problems associated with current HIFU treatments are due to the inability to control the amount of energy delivered and absorbed uniformly within a target treatment area. This results in an undesirable variability in tissue temperature. Some areas are over-treated, some under-treated, and some non-target areas are treated, all resulting in unacceptable outcomes. Embodiments of the present invention provide systems, devices, and methods for precisely controlling energy deposition throughout a target treatment area. Such energy application may be used for a variety of treatment purposes, including tissue ablation, precision hyperthermia, imaging, etc.

Embodiments described herein include individual needle probes, e.g., treatment probes that are embedded with one or more heating energy dispensing elements, such as small acoustic wave dispensing elements. The dispensing elements may be, e.g., acoustic transducers that convert electrical or mechanical energy into acoustic waves, or may be a lens (or lens assembly) that focuses acoustic waves generated from a separate wave generator. The needle probes may include a sharpened end so that they may penetrate the outer surface of an object, such as a patient's skin.

In some embodiments, a number of treatment probes may be positioned in an array to create a target treatment area all within the array volume. The spacing between treatment probes is not infinite in distance. Rather, spacing between treatment probes may be selected to ensure that uniform temperatures can be obtained at a target volume despite variable tissue types and conditions.

The amount of energy absorbed by the target volume may be precisely determined using a variety of techniques. In one embodiment, one or more temperature monitoring devices (e.g., thermistors, thermocouples, etc.) may be arranged proximate to or within the treatment volume. For example, one or more treatment probes may include a temperature monitoring device together with an acoustic wave dispensing element. For another example, one or more treatment probes may include a temperature monitoring device without any acoustic wave dispensing elements. For yet another example, the acoustic wave dispensing element may operate to measure the temperature of the treatment volume. In other embodiments, one or more temperature monitoring devices may be arranged external of the patient. For example, magnetic resonance imagers, infrared temperatures, externally applied ultrasound temperature sensors, and the like may be used. In some embodiments, instead of performing temperature monitoring, calculations may be performed to accurately estimate the temperature of the target volume. Such calculations may use factors such as target tissue type, characteristics of the acoustic wave dispensing element, distance of the acoustic wave dispensing element from the target tissue, orientation of the acoustic wave dispensing element with respect to the target tissue, and characteristics of the applied acoustic waves.

In at least one embodiment, real-time adjustments may be made to the amount of energy delivered based on either the temperature monitoring or calculated energy delivered. Accordingly, the temperature of the target volume may be used as feedback in controlling the amount of energy delivered.

By utilizing one or more of precision delivery of acoustic waves, precise high-resolution temperature monitoring, real-time control of and adjustment to the amount of energy delivered based on the temperature monitoring, and spacing of the individual treatment probes such that the energy may be uniformly delivered to a target volume resulting in achieving the temperature and/or energy absorption goal regardless of tissue composition and/or variability, one or more of the problems facing current HIFU delivery systems may be overcome.

System for Applying Heating Energy to a Target Volume

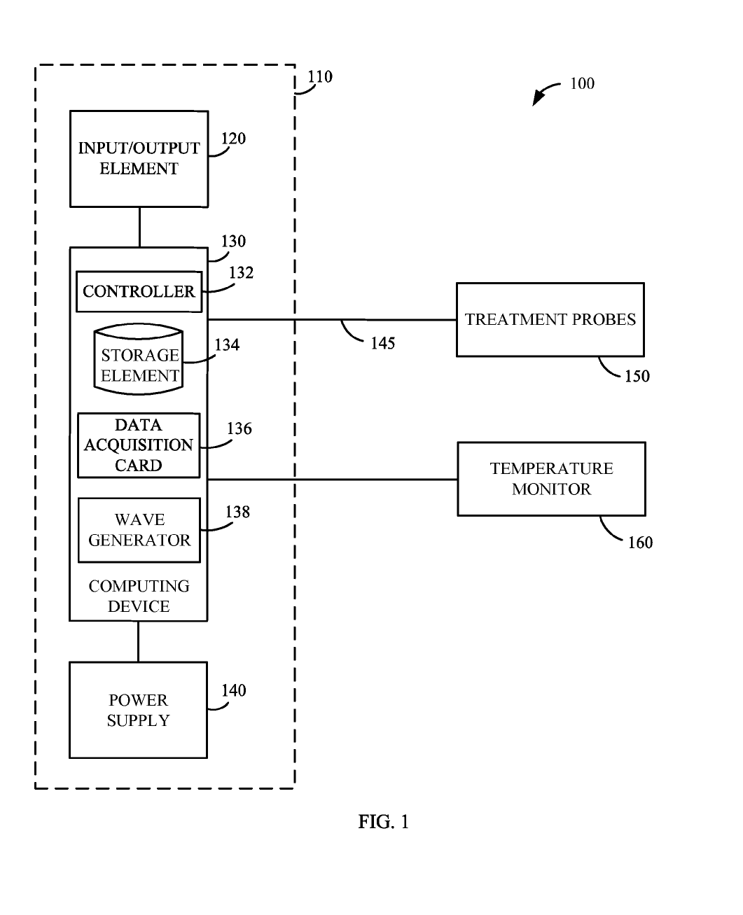

Turning to the figures, FIG. 1 is a block diagram of a simplified system 100 for selectively applying heating energy, such as acoustic waves, to target volumes in accordance with an embodiment. System 100 includes a system control unit 110 operatively coupled to treatment probes 150 and temperature monitor 160. System control unit 110 may include one or more elements, such as an input/output element 120, a computing device 130, and a power supply 140.

System control unit 110 may control treatment probes 150 to deliver heating energy (e.g., acoustic waves) to a target volume. In an acoustic waves embodiment, treatment probes 150 may be controlled to deliver acoustic waves at a variety of different intensities (e.g., 0.1-100 mW/cm² for diagnosis such as imaging and 100-10,000 W/cm² for therapy such as tissue ablation) and at a variety of different compression pressures (e.g., compression and rarefaction pressures of 0.001-0.003 MPa for diagnosis and peak compression pressures up to 30 MPa and peak rarefaction pressures up to 10 MPa for therapy).

System control unit 110 may be coupled to (or may include) temperature monitor 160, and use temperature monitor 160 to monitor the temperature of a target volume or calculate an estimated amount of energy absorbed by the target volume. In one embodiment, one or more monitors 160 (e.g., thermistors, thermocouples, etc.) may be arranged proximate to or within the treatment volume. For example, one or more of treatment probes 150 may include a temperature monitor 160 together with an acoustic wave dispensing element (not shown) arranged within treatment probes 150. For another example, one or more treatment probes 150 may include a temperature monitor 160 without any acoustic wave dispensing elements. For yet another example, the temperature monitor 160 may be an acoustic wave dispensing element. In other embodiments, one or more temperature monitors 160 may be arranged external of the patient. For example, temperature monitors 160 may include magnetic resonance imagers, infrared temperature sensors, externally applied ultrasound temperature sensors, and the like. In some embodiments, instead of performing temperature measurements, temperature monitor 160 may perform calculations to accurately estimate the temperature of the target volume. Such calculations may use factors such as target tissue type, characteristics of the acoustic wave dispensing element, distance of the acoustic wave dispensing element from the target tissue, orientation of the acoustic wave dispensing element with respect to the target tissue, and characteristics of the applied acoustic waves.

The elements of system control unit 110 may cooperatively configure the control unit to perform one or more of the operations discussed herein. Input/output element 120 may be any suitable device or devices for receiving inputs from an operator and providing outputs to the operator. For example, input/output element 120 may include a keyboard, a mouse, a keypad, a trackball, a light pen, a touch screen display, a non-touch screen display (e.g., a cathode ray tube display, a liquid crystal display, a light emitting diode display, a plasma display, etc.), a speaker, etc. Input/output element 120 may be operable to perform input/output functions as described herein, such as receiving a desired temperature input from the operator, receiving a selection of one or more desired treatment probes to activate, displaying a current temperature of a treatment volume to the operator, etc.

Computing device 130 may include, e.g., a computer or a wide variety of proprietary or commercially available computers or systems having one or more processing structures, a personal computer, and the like, with such systems often comprising data processing hardware and/or software configured to implement any one (or combination of) the processing operations described herein. Any software will typically include machine readable code of programming instructions embodied in a non-transitory tangible media such as an electronic memory, a digital or optical recovering media, or the like, and one or more of these structures may also be used to transmit data and information between components of the system in any wide variety of distributed or centralized signal processing architectures.

According to one embodiment, computing device 130 includes a controller 132 such as a single core or multi-core processor and a storage element 134 such as a tangible non-transitory computer-readable storage medium, where processor 132 may execute computer-readable code stored in storage element 134.

Computing device 130 may also include a data acquisition card 136. Data acquisition card 136 may be electrically or wirelessly coupled to treatment probes 150 and/or temperature monitor 160 so as to receive various measurement data from treatment probes 150 and/or temperature monitor 160. For example, data acquisition card 136 may receive temperature measurements from temperature sensors included in treatment probes 150, or temperature measurements from temperature monitor 160.

In some embodiments, computing device 130 may also include a wave generator 138. Wave generator 138 may be operable to generate acoustic waves. The acoustic waves may be in the ultrasound frequency band (e.g., 20 kHz to 200 MHz or greater than 200 MHz), the audible frequency band (e.g., 20 Hz to 20 kHz), or the infrasound frequency band (e.g., less than 20 Hz). The acoustic waves may have a variety of different intensities (e.g., 0.1-100 mW/cm² for diagnosis such as imaging and 100-10,000 W/cm² for therapy such as tissue ablation) and at a variety of different compression pressures (e.g., compression and rarefaction pressures of 0.001-0.003 MPa for diagnosis and peak compression pressures up to 30 MPa and peak rarefaction pressures up to 10 MPa for therapy).

System control unit 110 may also include power supply 140, which may be any suitable power supply for supplying power to input/output element 120 and/or computing device 130. In one embodiment, power supply 140 may include a power converter for converting AC power received from an AC power source (located external to system control unit 110) to DC power. In other embodiments, power supply 140 may include a battery.

System 100 also may include a communication element 145 coupled to treatment probes 150 and operable to communicate signals for controlling the acoustic waves output by treatment probes 150. In one embodiment, treatment probes 150 may include an acoustic transducer configured to convert electrical or mechanical signals to acoustic waves. In such cases, communication element 145 may be a wire or other electrical conductor that communicates electrical signals from computing device 130 to treatment probes 150. In another embodiment, treatment probes 150 may include an acoustic lens configured to focus or otherwise redirect acoustic waves to a treatment volume. In such cases, communication element 145 may be a waveguide or other element operable to communicate acoustic waves from wave generator 138 to the acoustic lens located in treatment probes 150. In yet other embodiments, communication element 145 may be a wireless communication channel (e.g., using RF communication, IR communication, or other wireless communication technique) operable to communicate control signals from computing device 130 to a wireless receiver located in treatment probes 150.

Treatment probes 150 includes one or more probes configured to pierce the outer surface of an object to reach a treatment volume. At least one of the probes includes an acoustic wave dispensing element operable to output acoustic waves. In some embodiments, an array of elongated probes may be provided. The probes may output acoustic waves based on signals (electrical, acoustic, etc.) communicated from computing device 130. In some embodiments, one or more probes may include or be replaced by a temperature monitor (e.g., a thermistor, a thermocouple, etc.) for measuring a temperature of the probe or within a vicinity of the probe (e.g., at a target volume). In at least one embodiment, one or more treatment probes 150 may also or alternatively acquire images of the treatment volume. For example, a treatment probe may acquire images using the acoustic waves output by the treatment probe.

According to one embodiment, the treatment probes may be individually advanced and positioned within a target tissue (e.g., a prostate). Once the probes are positioned, one or more ultrasonic waves may be applied to the target tissue via the probes, thereby causing the target tissue to absorb energy and increase in temperature. Such waves may be used, for example, for tissue ablation, hyperthermia, imaging, etc.

System 100 in certain embodiments is a system for selectively applying acoustic waves to target volumes, and includes various components such as an input/output element 120, a computing device 130, and a power supply 140. However, it will be appreciated by those of ordinary skill in the art that system 100 could operate equally well by having fewer or a greater number of components than are illustrated in FIG. 1. Thus, the depiction of system control unit 108 in FIG. 1 should be taken as being illustrative in nature, and not limiting to the scope of the disclosure.

Arrangement of Treatment Probes Proximate a Target Volume

As described herein, a number of treatment probes may be positioned in a target treatment area all, and may include an array of treatment probes. The spacing between treatment probes may be selected to ensure that a more even or uniform distribution of temperatures can be obtained at a target volume despite variable tissue types and conditions, and/or to more precisely control heating energy and selected heating to the target tissue within a desired temperature range. Embodiments are described with reference to acoustic wave heating energy delivery, though the described structures and methods shall not be limited solely to one heating energy embodiment.

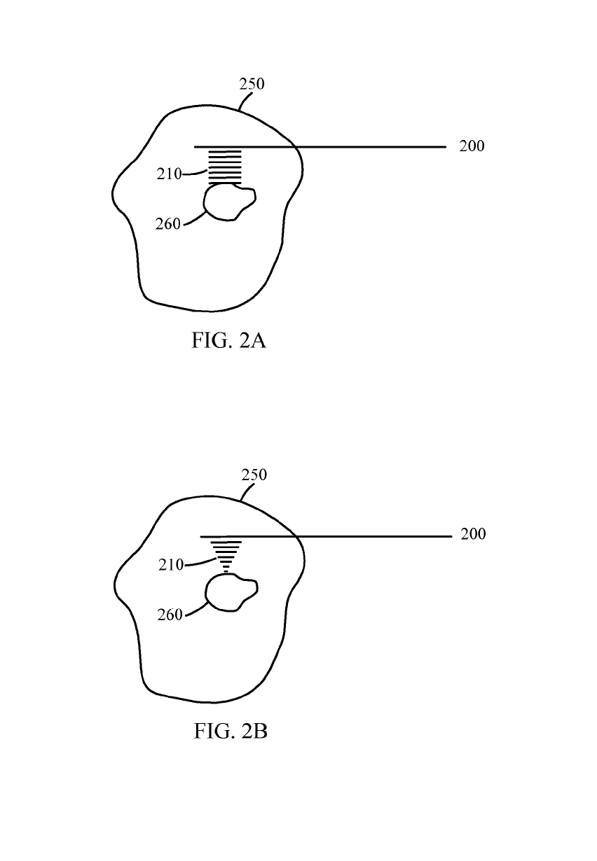

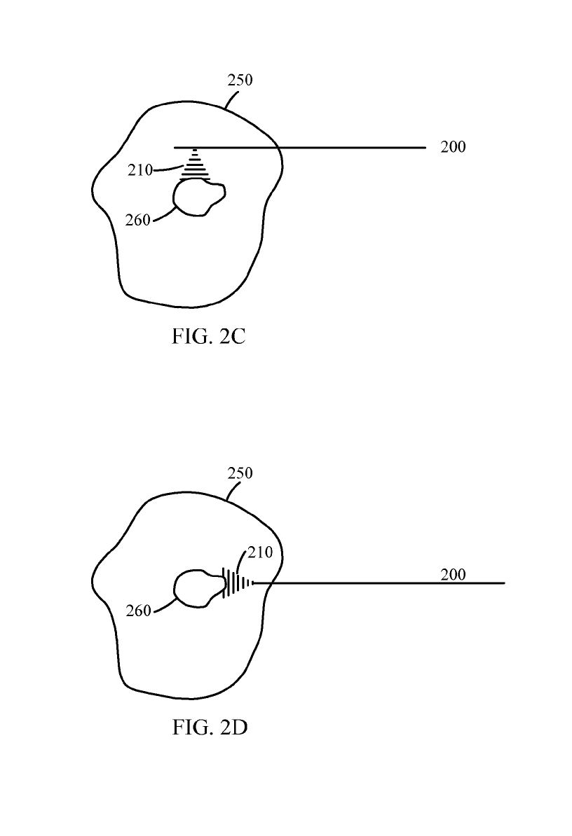

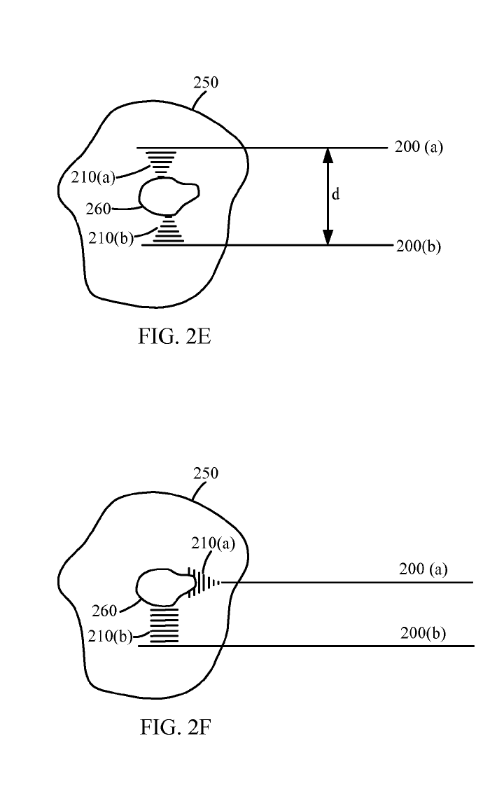

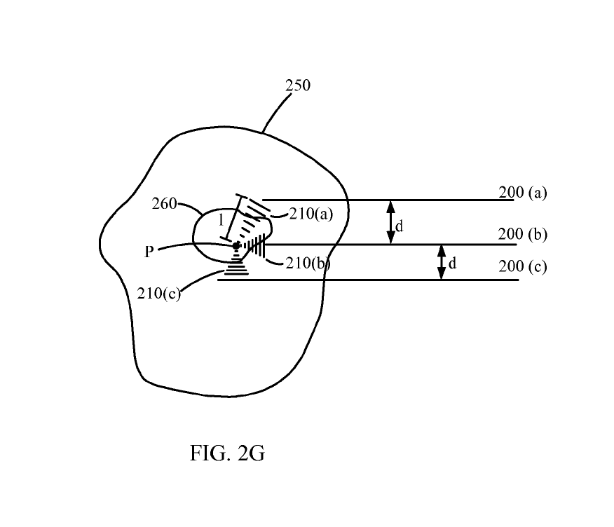

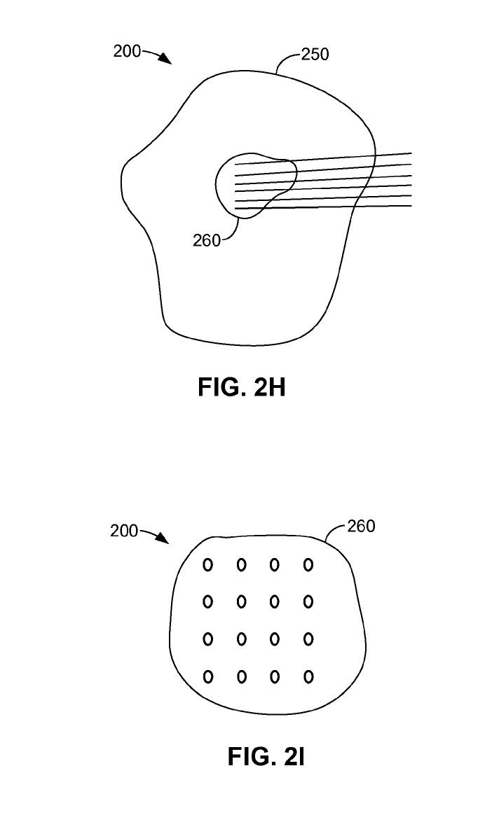

FIGS. 2A to 2I illustrate treatment probes applying heating energy, e.g., acoustic waves, to target volumes in accordance with numerous embodiments. The treatment probes may be configured to output acoustic waves from any suitable location of the probe, such as from an end, from a longitudinal surface, or from other locations. The acoustic waves may be output at any suitable angle from the probe, such as an angle that is perpendicular to a longitudinal axis of the probe, parallel to the longitudinal axis of the probe, or somewhere in between. The acoustic waves may be focused waves or, in other embodiments, may be transverse waves, dispersed waves, or have other propagation characteristics. The probes may be inserted into an object (such as a patient) and located to focus acoustic waves on a target volume. Where a number of probes are used, they may be spaced apart from one another and configured so as to focus acoustic waves on a target volume from a number of different directions.

Turning to FIG. 2A, FIG. 2A illustrates a treatment probe 200 applying acoustic waves 210 to a target volume 260 located in an object 250 in accordance with an embodiment. Object 250 may be any object for which it is desired to apply acoustic waves to a target volume 260 located therein. In one embodiment, object 250 may be a patient and target volume 260 may be tissue arranged within the patient. In other embodiments, object 250 may be a metal, polymer, ceramic, or other type of material, and may be in a solid, liquid, or other suitable state.

In accordance with the embodiment depicted in FIG. 2A, treatment probe 200 is configured to output an acoustic wave 210 that is a transverse wave in a direction perpendicular to a longitudinal direction of probe 200. In FIG. 2B, treatment probe 200 is configured to output an acoustic wave 210 similar to that described with reference to FIG. 2A, except that in this embodiment acoustic wave 210 is a focused wave. In FIG. 2C, treatment probe 200 is configured to output an acoustic wave 210 similar to that described with reference to FIG. 2A, except that in this embodiment acoustic wave 210 is a dispersed wave.

In accordance with the embodiment depicted in FIG. 2D, treatment probe 200 is configured to output an acoustic wave 210 that is a transverse wave in a direction parallel to a longitudinal direction of probe 200. One skilled in the art would recognize that the different types of waves described with reference to FIGS. 2A to 2C could similarly be used in the embodiment depicted in FIG. 2D. Further, one skilled in the art would recognize that acoustic wave 210 need not be output in a direction that is perpendicular or parallel to a longitudinal direction of probe 200, but could be output at any other suitable angle with reference to the longitudinal direction of probe 200 (e.g., at a direction between the directions perpendicular and parallel to the longitudinal direction of probe 200).

FIG. 2E illustrates a first treatment probe 200(a) and a second treatment probe 200(b), where first treatment probe 200(a) is operable to apply a first acoustic wave 210(a) to target volume 260 and second treatment probe 200(b) is operable to apply a second acoustic wave 210(b) to target volume 260. In this embodiment, first acoustic wave 210(a) and second acoustic wave 210(b) are of the same wave type, i.e., they are both focused waves. However, in other embodiments, they may have different wave types. Further, in this embodiment, they are spaced apart from one another by a distance d such that the distance from each probe to target volume 260 is the same, and they are embedded to the same depth within object 250. Such a configuration of probes may be advantageous as the same probes can be used in the array of probes and simply rotated 180 degrees with respect to one another to attain a common target volume.

The embodiment illustrated in FIG. 2F is similar to that described with reference to FIG. 2E, however in this case different types of acoustic waves are generated and they are applied at different angles. First treatment probe 200(a) outputs a first acoustic wave 210(a) that is a dispersion wave output at an angle that is parallel to the longitudinal direction of first treatment probe 200(a), whereas second treatment probe 200(b) outputs a second acoustic wave 210(b) that is a transverse wave output at an angle that is perpendicular to the longitudinal direction of second treatment probe 200(b). Further, the treatment probes are embedded to different depths within object 250. One skilled in the art would recognize various other combinations, and all such combinations are within the scope of this disclosure.

FIG. 2G illustrates a first treatment probe 200(a), a second treatment probe 200(b), and a third treatment probe 200(c), where first treatment probe 200(a) is operable to apply a first acoustic wave 210(a) to target volume 260, second treatment probe 200(b) is operable to apply a second acoustic wave 210(b) to target volume 260, and third treatment probe 200(c) is operable to apply a third acoustic wave 210(c) to target volume 260. In this embodiment, the acoustic waves are all focused waves that focus on a point P in target volume 260. The acoustic waves each have a focal length 1 that is equal to the distance from the acoustic wave dispensing element (not shown) in the probe to the focal point of the acoustic wave. In this embodiment, the focal length of each probe is different, the output angle of the acoustic waves is different, and the distance d between probes is different. However, these characteristics are configured such that the acoustic waves output from the treatment probes all focus on a point P in target volume 260. In other embodiments, some or all of these characteristics may be the same, as long as the acoustic waves output from the treatment probes all focus on a point P in target volume 260.

One or more treatment probes 200 may be disposed within an object 250 to apply acoustic waves to a target volume 260 located in the object 250. The one or more treatment probes 200 can include an array of treatment probes, e.g., as conceptually illustrated with reference to FIGS. 2H and 2I. FIG. 2H shows an array of treatment probes 200 disposed in an object 250 and treatment volume 260. FIG. 2I illustrates a frontal cross-section view of a treatment volume 260 having an array of treatment probes 200 disposed therein.

While various embodiments are depicted, illustrating various wave types, wave angles, wave focal lengths, distance between probes, and the like, it will be appreciated by those of ordinary skill in the art that the arrangements disclosed herein are not limited to those explicitly illustrated in FIGS. 2A to 2I. Furthermore, while certain embodiments are illustrated as including acoustic waves, various types of heating energies may be employed (e.g., acoustic, laser, infrared, ionizing radiation, and the like—see also, below). Rather, the scope of the disclosure includes various combinations of the characteristics described herein. Thus, the depiction of treatment probes in FIGS. 2A to 2I should be taken as being illustrative in nature, and not limiting to the scope of the disclosure.

Characteristics of Treatment Probes

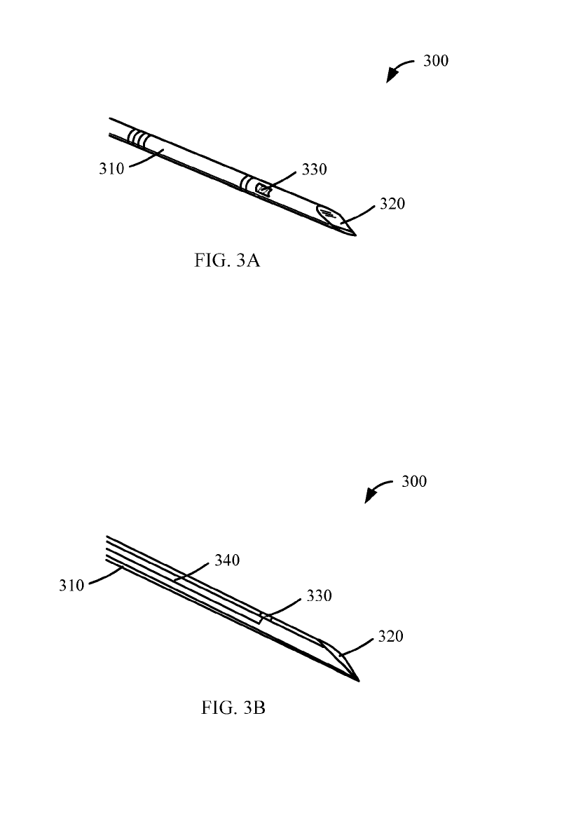

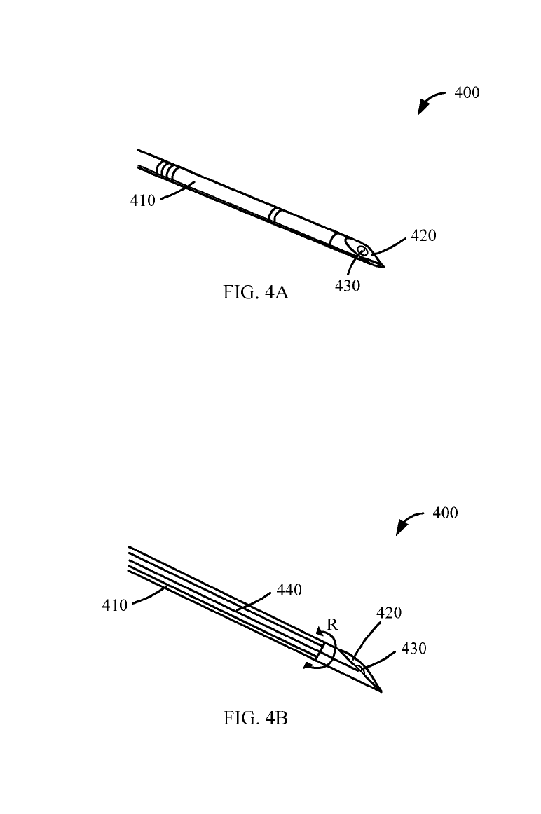

FIGS. 3A to 4B illustrate profile views and cross-sectional views of treatment probes according to various embodiments of the invention. The treatment probes may include a piercing end, where the piercing end is sharpened so as to penetrate an outer surface of an object (e.g., the skin of a patient). The probes include acoustic wave dispensing elements arranged at various locations on the probes, and include communication elements coupled to the acoustic wave dispensing elements operable to communicate signals for controlling acoustic waves output by the acoustic wave dispensing elements.

Turning to FIG. 3A, FIG. 3A is a profile view of a treatment probe 300 according to an embodiment. Treatment probe 300 includes a housing 310, a piercing end 320, and an a heating energy dispensing element such as acoustic wave dispensing element 330. Housing 310 is configured to support acoustic wave dispensing element 330 and, in one embodiment, is elongated and has a cylindrical shape. However, housing 310 may form or include other shapes as well. The housing 310 includes a piercing end 320 that is configured to pierce through an outer surface of an object, such as exposed skin of a patient. Housing 310 and/or piercing end 320 may be made of any suitable material sufficiently strong to pierce the outer surface of the object. For example, piercing portion 310 may be made of metal, ceramic, composite materials, etc.

Acoustic wave dispensing element 330 is coupled to housing 310 and is operable to output acoustic waves. Acoustic wave dispensing element 330 according to this embodiment is arranged on an outer surface of housing 310, and may output acoustic waves at an angle perpendicular to the longitudinal direction of housing 310.

FIG. 3B is a cross-sectional view of the probe of FIG. 3A. From the cross-sectional view, various components of a probe according to one embodiment are visible. According to this embodiment, probe 300 includes a communication element 340 coupled to acoustic wave dispensing element 330 and operable to communicate signals for controlling the acoustic waves. Acoustic wave dispensing element 330 may, for example, be an acoustic transducer, or may, for example, be a lens. Communication element 340 may, for example, be an electrical conductor, or may, for example, be a waveguide.

FIG. 4A is a profile view of a treatment probe 400 according to a second embodiment, and FIG. 4B is a cross-sectional view of the treatment probe 400 of FIG. 4A. Treatment probe 400 is similar to treatment probe 300 described with reference to FIGS. 3A and 3B, and reference numbers 410 through 440 are correspondingly similar to reference numbers 310 through 330.

In the embodiments depicted in FIGS. 4A and 4B, however, acoustic wave dispensing element 330 is arranged on an angled surface of piercing end 420. Further, piercing end 420 may be rotated along a direction R so as to alter a direction from which acoustic waves are output from acoustic wave dispensing element. Piercing end 420 may be rotated using any suitable mechanism, including mechanical, electrical, and/or wireless mechanisms. For example, communication element 340 may also include control signals for controlling the rotation of piercing end 420.

Probes 300 and 400 in certain embodiments may include various components such as a housing, a piercing end, an acoustic wave dispensing element, and a communication element. However, it will be appreciated by those of ordinary skill in the art that the probes could operate equally well by having fewer or a greater number of components than are illustrates in FIGS. 3A through 4B. Thus, the depiction of probes 300 and 400 in FIGS. 3A through 4B should be taken as illustrative in nature, and not limiting to the scope of the disclosure.

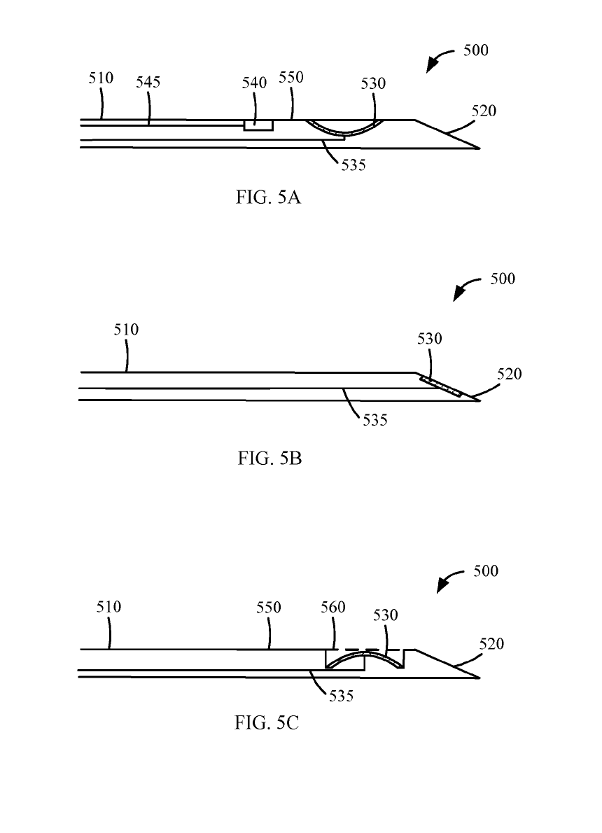

FIGS. 5A to 5C are cross-sectional views of treatment probes including acoustic transducers according to numerous embodiments. The treatment probes may be configured to output acoustic waves using acoustic transducers. The acoustic transducers may be provided on any suitable surface of the probe so as to direct acoustic waves in a variety of different directions. Further, the acoustic transducers may be shaped to generate focused waves, transverse waves, dispersion waves, or other wave types suitable to treat a target volume. In some embodiments, the treatment probes may also include a temperature monitor (i.e., a temperature sensor), that monitors a temperature of the probe or in the vicinity of the probe (e.g., at a target volume). Further, in some embodiments, a direction and focal depth of the output acoustic waves is constant, whereas in other embodiments the direction and/or focal depth of the output acoustic waves is variable.

FIG. 5A is a cross-sectional view of a treatment probe 500 according to an embodiment. Treatment probe 500 includes a housing 510, a piercing end 520, an acoustic wave dispensing element 530, and a temperature monitor (e.g., a temperature sensor) 540. Housing 510, piercing end 520, and acoustic wave dispensing element 530 are similar to the corresponding elements 310 to 330 described with reference to FIG. 3. However, in this embodiment, acoustic wave dispensing element 530 is a concave acoustic transducer configured to generate focused acoustic waves in response to an electrical, mechanical, or other stimulus. The acoustic transducer may, e.g., be an electromagnetic acoustic transducer, a piezoelectric acoustic transducer, or other suitable transducer for generating acoustic waves.

Acoustic wave dispensing element 530 according to this embodiment is arranged on a surface of probe 500 other than piercing end 520, and is configured to output acoustic waves in a direction perpendicular to the longitudinal axis of probe 500. In other embodiments, acoustic wave dispensing element 530 may be arranged on different surfaces of probe 500, and/or may be configured to output acoustic waves in directions other than a direction perpendicular to the longitudinal axis of probe 500. Further, in this embodiment acoustic wave dispensing element 530 is configured to be embedded within probe 500 such that it is retained within an outer surface 550 of probe 500. Such an arrangement may advantageously reduce damage to the object in which probe 500 is disposed for treatment.

Probe 500 may also include a communication element 535 coupled to acoustic wave dispensing element 530 and extending within housing 510 and along a length of probe 500. In this embodiment, communication element 535 is a conductive wire (for electrically or magnetically actuating acoustic transducer 530), a resilient member (for mechanically actuating acoustic transducer 530), or other suitable component for actuating acoustic transducer 530. In some embodiments, communication element 535 may be operable to communicate signals resulting from actuation of acoustic transducer 530. For example, when acoustic transducer 530 is used for imaging or measuring temperature, acoustic transducer 530 may be actuated from acoustic signals reflected from a target volume, and signals indicative of such actuation may be communicated from transducer 530 via communication element 535.

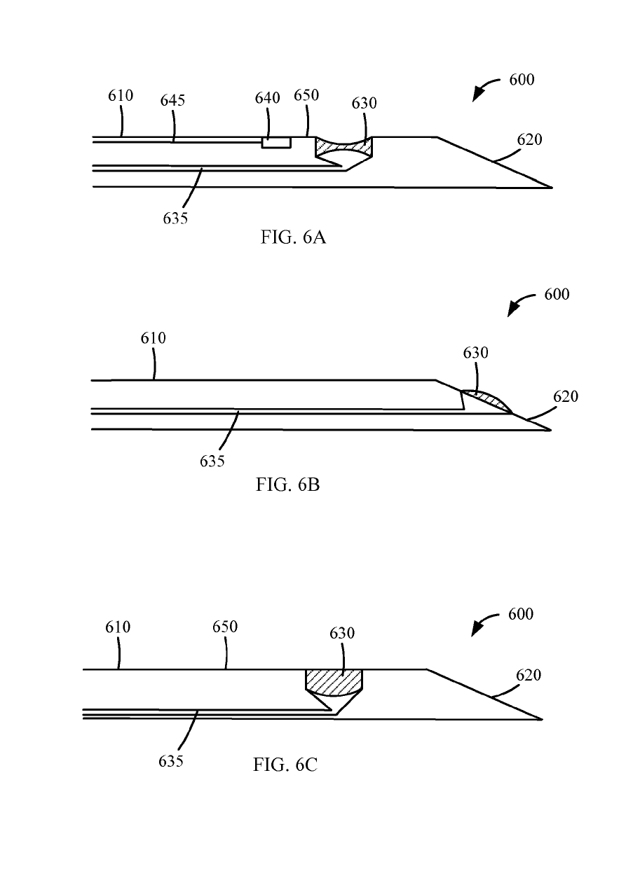

Temperature monitor 540 may be any suitable component for measuring temperature, such as a thermistor, a thermocouple, etc. Temperature monitor 540 according to this embodiment is arranged on a surface of probe 500 other than piercing end 520, and is configured to monitor temperature at a location proximate to the longitudinal axis of probe 500. In other embodiments, temperature monitor 540 may be arranged on different surfaces and/or different locations of probe 500, such as at piercing end 520, and may be arranged on different probes such as any of those described with reference to FIGS. 5B to 6C. Further, in this embodiment temperature monitor 540 is configured to be embedded within probe 500 such that it is retained within outer surface 550. Such an arrangement may advantageously reduce damage to the object in which probe 500 is disposed for treatment.

Probe 500 may also include a communication element 545 coupled to temperature monitor 540 and extending within housing 510 and along a length of probe 500. In this embodiment, communication element 545 is a conductive wire (for electrically or magnetically communicating signals indicative of temperature from temperature monitor 540).

In some embodiments, probe 500 may also include wireless communication circuitry (not shown). Such circuitry may be operable to communicate temperature signals from temperature monitor 540, control signals to acoustic transducer 530, and/or signals resulting from actuation of acoustic transducer 530, as previously described.

FIG. 5B is a cross-sectional view of a treatment probe 500 according to another embodiment. Treatment probe 500 is similar to that described with reference to FIG. 5A, however in this embodiment acoustic wave dispensing element 530 is arranged at piercing end 520 and is configured to output acoustic waves at an angle relative to the longitudinal axis of housing 510. Further, acoustic wave dispensing element 530 is a planar acoustic transducer, thereby facilitating the generation of transverse acoustic waves.

FIG. 5C is a cross-sectional view of a treatment probe 500 according to yet another embodiment. Treatment probe 500 is similar to that described with reference to FIG. 5A, however in this embodiment acoustic wave dispensing element 530 is a convex acoustic transducer, thereby facilitating the generation of dispersion waves. Further, in some embodiments, treatment probe 500 may include an acoustically transparent window 560 that is transparent to acoustic waves generated by and/or reflected back toward acoustic wave dispensing element 530. Transparent window 560 may be flush with outer surface 550, and acoustic wave dispensing element 530 may be arranged behind window 560. In this fashion, acoustic wave dispensing element 530 may be embedded within probe 500 and have a variety of shapes, while outer surface 550 may be smooth so as to reduce damage to the object in which probe 500 is disposed for treatment.

FIGS. 6A to 6C are cross-sectional views of treatment probes including acoustic lenses according to numerous embodiments. The treatment probes may be configured to output acoustic waves using acoustic lenses and waveguides. The acoustic lenses may be provided on any suitable surface of the probe so as to direct acoustic waves in a variety of different directions. Further, the acoustic lenses may be shaped to generate focused waves, transverse waves, dispersion waves, or other wave types suitable to treat a target volume. In some embodiments, the treatment probes may also include a temperature monitor (i.e., a temperature sensor), that monitors a temperature of the probe or in the vicinity of the probe (e.g., at a target volume). Further, in some embodiments, a direction and focal depth of the output acoustic waves is constant, whereas in other embodiments the direction and/or focal depth of the output acoustic waves is variable.

FIG. 6A is a cross-sectional view of a treatment probe 600 according to an embodiment. Treatment probe 600 is similar to probe 500 described with reference to FIG. 5A, where elements 610 through 650 correspond to elements 510 through 550. However, in this embodiment acoustic wave dispensing element 630 is an acoustic lens. The acoustic lens according to this embodiment is a thin lens, however in other embodiments different types of acoustic lenses may be used, such as a Fresnel lens, a spherical lens (using one or more acoustically conductive spheres), a plate lens (slant-plate lens, perforated-plate lens, etc.), a thick lens, a compound lens, a cylindrical lens, etc. Acoustic lens 630 in this embodiment is configured to focus acoustic waves communicated to lens 630 via communication element 635. Communication element 635 is a waveguide or other element operable to communicate acoustic waves from a wave generator (arranged within or external to probe 600) to acoustic lens 630.

FIG. 6B is a cross-sectional view of a treatment probe 600 according to another embodiment. Treatment probe 600 is similar to that described with reference to FIG. 6A, however in this embodiment acoustic wave dispensing element 630 is arranged at piercing end 620 and is configured to output acoustic waves at an angle relative to the longitudinal axis of housing 610. Further, acoustic wave dispensing element 630 may be a lens shaped to correct variations in the direction of acoustic waves caused by a shape of communication element 635, thereby facilitating the generation of transverse acoustic waves.

FIG. 6C is a cross-sectional view of a treatment probe 600 according to yet another embodiment. Treatment probe 600 is similar to that described with reference to FIG. 6A, however in this embodiment acoustic wave dispensing element 630 is a thick lens, thereby facilitating the generation of dispersion waves as the focal point may be located within lens 630.

Probes 500 and 600 in certain embodiments may include various components such as acoustic transducers, acoustic lenses, temperature monitors, etc. However, it will be appreciated by those of ordinary skill in the art that the probes could operate equally well by having fewer or a greater number of components than are illustrated in FIGS. 5A through 6C. Thus, the depiction of probes 500 and 600 in FIGS. 5A through 6C should be taken as illustrative in nature, and not limiting to the scope of the disclosure.

For example, in some embodiments the focal point of an acoustic wave dispensing element may be variable. A variable focal point may be achieved using any one or more of a number of techniques. For example, where acoustic transducers are used, the transducer may be made of flexible semiconductor material, a number of movable transducers having converging focal points may be used, etc. The semiconductor material may be flexed or the transducers moved in response to pressure applied from a mechanical actuator, or by some other mechanism. For another example, where acoustic lens are used, a variable focus lens assembly may be used (changing a distance between lens, changing a lens shape arranged at an interface between two liquid cavities, changing the electrical voltage applied to a multi-layer liquid crystal lens, changing the shape of a liquid drop in a multi-liquid lens, etc.). The focal point of the acoustic wave dispensing element may be controlled by any suitable entity. For example, computing device 130 (FIG. 1) may send control signals to the acoustic wave dispensing element via, e.g., a communication element similar to those described herein, so as to control the focal point of the acoustic wave dispensing element.

Application of Energy with Precision Temperature Monitoring

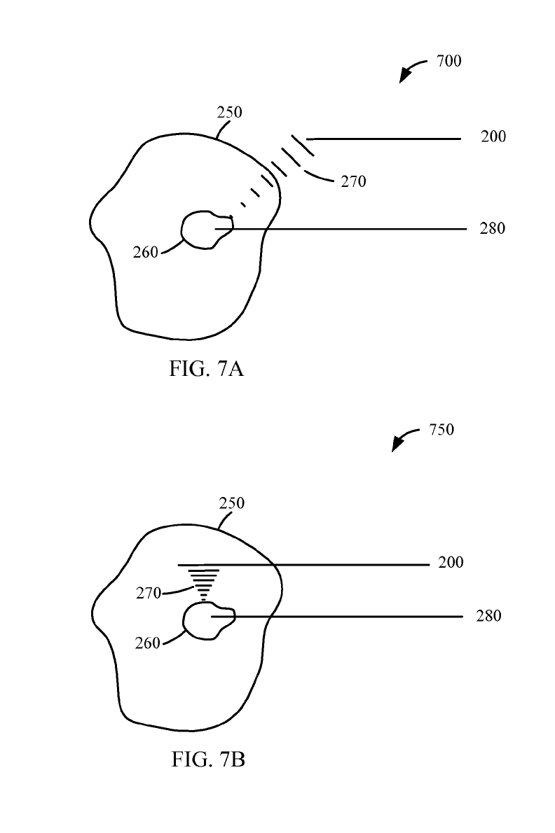

FIGS. 7A and 7B illustrate treatment probes applying energy to target volumes while the temperature of the target volumes is precisely monitored. In one embodiment, energy is applied using a treatment probe that is external to the patient and target volume, while in another embodiment energy is applied using a treatment probe that is disposed in the patient. The energy may be in the form of acoustic waves, or the applied energy may take a different form, such as electromagnetic waves in one or more frequency bands, such as radio waves, microwaves, infrared waves, visible light waves, ultraviolet waves, x-rays, gamma rays, etc. The temperature of the target volume is precisely monitored and used to control the amount and/or type of energy applied. In these embodiments, the temperature of the target volume is monitored using probes having temperature sensors, where the temperature sensors are located in the target volume. The amount of energy applied to the target volume may be controlled using the temperature of the target volume such that the temperature of the target volume is maintained at a desired temperature for a set period of time. The desired temperature may be sufficient for ablating tissue of the target volume (e.g., temperatures above 60 degrees Celcius for periods of 1 second, 5 seconds, 10 seconds, or 15 seconds), causing hyperthermia (e.g., temperatures approximately equal to 43 degrees Celcius for approximately one hour), or causing mild hyperthermia (e.g., temperatures in the range of 41 degrees Celcius to 43 degrees Celcius).

Turning to the figures, FIG. 7A illustrates a treatment probe 200 externally applying energy 270 to a target volume 260 located in an object 250 while a temperature probe 280 is disposed in the target volume 260. Treatment probe 200 may take the form of any of the probes described herein. For example, treatment probe 200 may include an acoustic transducer or acoustic lens for outputting acoustic waves to target volume 260. In other embodiments, treatment probe 200 may output electromagnetic waves in one or more frequency bands, such as radio waves, microwaves, infrared waves, visible light waves, ultraviolet waves, x-rays, gamma rays, etc. Treatment probe 200 is arranged outside of the object 250 in this embodiment. For example, treatment probe 200 may be arranged external to a patient. In this case, the energy transmitted from treatment probe 200 may travel through portions of object 250, including an outer surface of object 250, prior to reaching target volume 260. In at least one embodiment, the energy communicated from treatment probe 200 may be focused such that the focal point of the energy is at the treatment volume.

Temperature probe 280 may also take the form of any of the probes described herein, where temperature probe 280 includes at least one temperature sensor. For example, temperature probe 280 may be a probe including temperature sensor 540/640 (FIGS. 5A and 6A), but excluding an acoustic transducer and acoustic lens. For another example, temperature probe 280 may include an acoustic transducer that operates to measure temperature of the target volume. In at least one embodiment, temperature probe 280 is temperature monitor 160 (FIG. 1), and operates to provide temperature measurements of the target volume to system control unit 110 (FIG. 1).

Turning to the figures, FIG. 7B is similar to FIG. 7A, except in this case illustrates a treatment probe 200 internally applying energy 270 to a target volume 260 located in an object 250 while a temperature probe 280 is disposed in the target volume 260. Treatment probe 200 is arranged inside of the object 250 in this embodiment. For example, treatment probe 200 may be arranged internal to a patient. In this case, the energy transmitted from treatment probe 200 may travel through minimal portions of object 250 prior to reaching target volume 260. Like the embodiment described with reference to FIG. 7B, in this embodiment, temperature probe 280 may also take the form of any of the probes described herein, where temperature probe 280 includes at least one temperature sensor.

It should be recognized that embodiments are not limited to providing single treatment probes and temperature probes. Rather, in some embodiments, a number of treatment probes may be used, internally and/or externally, to apply energy to one or more treatment volumes. Similarly, one or more temperature probes may be used to monitor the temperature of the treatment volumes. In one particular embodiment, one temperature probe may be provided for each treatment probe, and disposed to monitor the temperature of the target volume of the associated treatment probe.

Methods for Treating a Target Volume Using Acoustic Waves

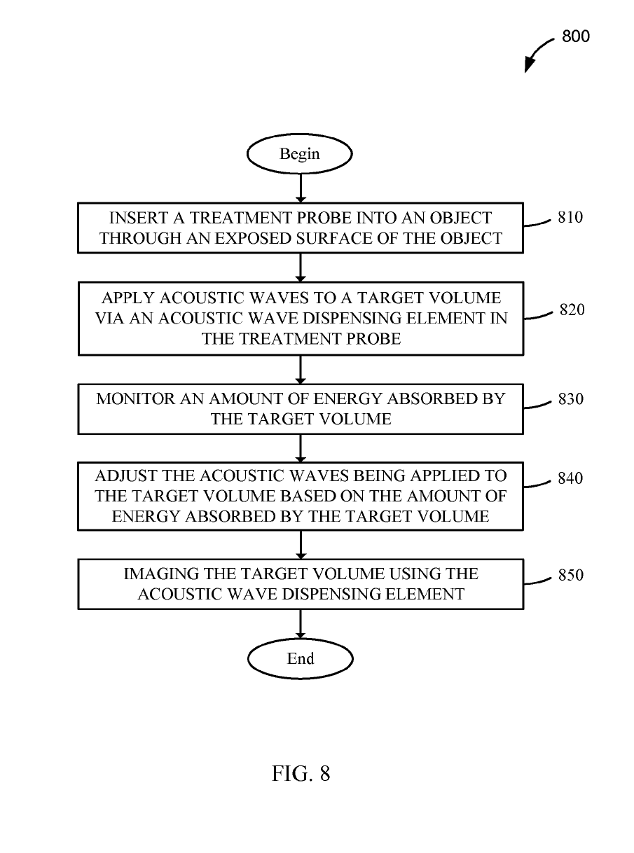

FIG. 8 is a flowchart 800 depicting example operations of a method for treating a target volume using acoustic waves according to a first embodiment. The acoustic waves may be communicated to the target volume using any suitable acoustic wave dispensing element, including any of those described with reference to FIGS. 2A through 6C. Further, the acoustic waves may be communicated from a source located external or internal to an object including the target volume, and a temperature of the target volume may be monitored using a temperature probe, as depicted in and described with reference to FIGS. 7A and 7B.

In operation 810, a treatment probe is inserted into an object through an exposed surface of the object. For example, a treatment probe may be inserted through the exposed skin of a patient such that an acoustic wave dispensing element of the treatment probe is located proximate to a treatment volume. The treatment probe may be inserted at various depths to reach the treatment volume. In some embodiments, a plurality of treatment probes may be inserted into the object, where the treatment probes are spaced apart from one another such that they are all located proximate to a treatment volume. For example, the treatment probes may be spaced apart such that upon being disposed in the object, the acoustic wave dispensing elements of the probes are located equidistant from a treatment volume. In an alternative embodiment, instead of being inserted into the object, the treatment probe(s) may be disposed outside of the object, as depicted in and described with reference to FIG. 7A.

In operation 820, acoustic waves are applied to a target volume via an acoustic wave dispensing element in the treatment probe. For example, with reference to FIG. 1, controller 132 may send control signals to an acoustic transducer provided in one or more treatment probes 150, where the control signals control a frequency, intensity, and/or duration of acoustic waves generated by the acoustic transducer. For another example, controller 132 may control wave generator 138 to generate acoustic waves that are propagated to an acoustic lens included in one or more treatment probes 150 via a waveguide.

In operation 830, an amount of energy absorbed by the target volume is monitored. In one embodiment and with reference to FIG. 1, temperature monitor 160 may monitor a temperature at a target volume, such as the temperature of the focal point of an acoustic wave. Where a number of different treatment probes are used, temperature monitor 160 may monitor the temperature of the target volume associated with each treatment probe. In some embodiments, temperature monitor 160 may be external to the object, and may be, e.g., a magnetic resonance imager, an infrared temperature sensor, an ultrasound temperature sensor, or other external temperature sensing device. Temperature monitor 160 may measure the temperature at the target volume of each treatment probe in real-time, where temperature measurements may be received by data acquisition card 136. In other embodiments, temperature monitor 160 may be arranged internally to the object. For example, temperature monitor 160 may be a temperature sensor such as temperature sensor 540/640, and data acquisition card 136 may be operable to receive temperature measurements from the temperature sensor. Temperature monitor 160 may be disposed in the target volume as depicted in and described with reference to FIGS. 7A and 7B. For another example, an acoustic wave dispensing element may be used to measure the temperature of a target volume. For example, an acoustic wave dispensing element located in one or more treatment probes 150 may output an acoustic wave, receive a reflection indicative of target volume temperature, and communicate either the reflection or a signal corresponding to the reflection to data acquisition card 136.

In another embodiment, temperature monitor 160 may estimate the temperature at the target volume. For example, controller 132 may estimate the temperature using one or more of a variety of factors, such as the type of material of the target volume (e.g., target tissue type), characteristics of the acoustic wave dispensing element (e.g., loss characteristics, focal depth, etc.), distance of the acoustic wave dispensing element from the target volume, orientation of the acoustic wave dispensing element with respect to the target issue, and characteristics of the controlled output acoustic waves (e.g., intensity, compression pressure, rarefaction pressure, etc.).

In operation 840, the acoustic waves being applied to the target tissue volume are adjusted based on the amount of energy absorbed by the target volume. The amount of energy absorbed may be determined using, e.g., a temperature monitor and/or a calculated temperature estimate as previously described. The acoustic waves may be adjusted in one or more of a number of different ways. For example, the intensity, compression pressure, rarefaction pressure, focal depth, and/or direction of the acoustic waves may be adjusted. In some embodiments, the acoustic waves may be adjusted so as to achieve a desired target volume temperature.

In operation 850, the target volume is imaged using the acoustic wave dispensing element. For example, acoustic wave dispensing elements of one or more treatment probes 170 may be controlled to output acoustic waves having characteristics appropriate for imaging the target volume. In one embodiment, the acoustic waves may be controlled to have an intensity in the range of 0.1-100 mW/cm², and compression and rarefaction pressures in the range of 0.001-0.003 MPa. The same or different acoustic wave dispensing elements may be used to receive acoustic waves reflected from the target volume, and send information indicative of the reflected acoustic waves to data acquisition card 136.

It should be appreciated that the specific operations illustrated in FIG. 8 provide a particular method of treating a target volume using acoustic waves, according to certain embodiments of the present invention. Other sequences of operations may also be performed according to alternative embodiments. Alternative embodiments of the present invention may also perform the operations outlined above in a different order. Moreover, the individual operations illustrated in FIG. 8 may include multiple sub-operations that may be performed in various sequences as appropriate to the individual operation. Furthermore, additional operations may be added or existing operations removed depending on the particular applications. For example, in some embodiments, treatment may comprise only imaging the target volume as described with reference to operation 850. In other embodiments, treatment may not require imaging. In yet other embodiments, the amount of energy absorbed by the target volume may not be monitored, and/or the acoustic waves being applied to the target volume may not be adjusted. Further, in some embodiments, the acoustic waves may be adjusted in accordance with an algorithm stored in storage element 134, where such algorithm may or may not use inputs from a monitored amount of energy absorbed by the target volume. Further, in some embodiments, heating of the target volume may be limited to application of acoustic waves, but may include the application of other forms of energy, such as electromagnetic waves. One of ordinary skill in the art would recognize and appreciate many variations, modifications, and alternatives.

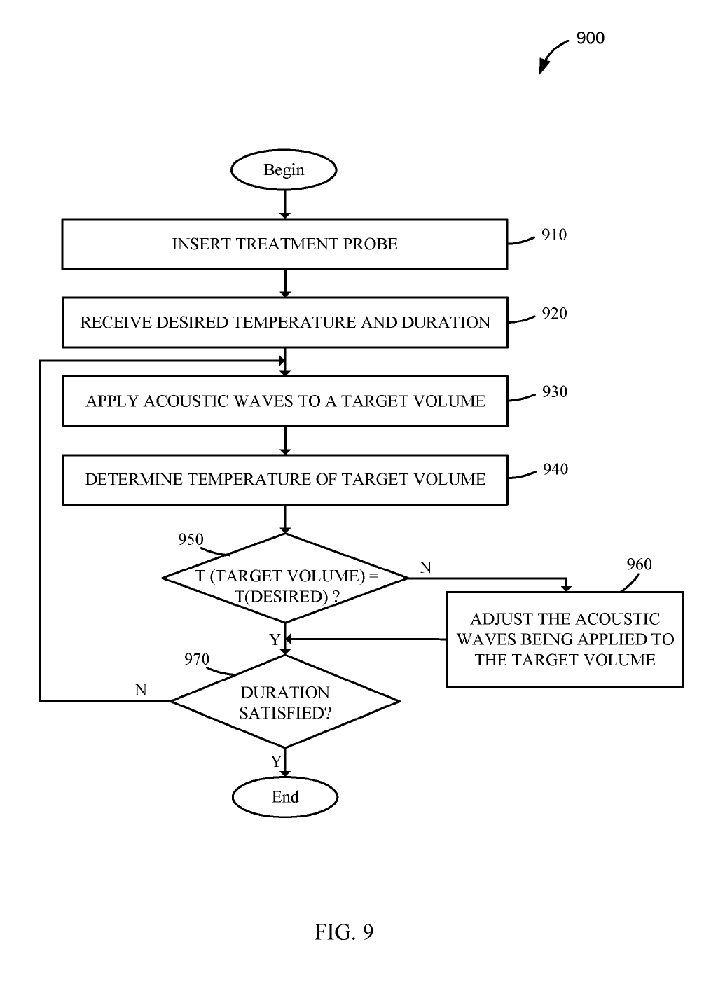

FIG. 9 is a flowchart 900 depicting example operations of a method for treating a target volume using acoustic waves according to a second embodiment. The acoustic waves may be communicated to the target volume using any suitable acoustic wave dispensing element, including any of those described with reference to FIGS. 2A through 6C. Further, the acoustic waves may be communicated from a source located external or internal to an object including the target volume, and a temperature of the target volume may be monitored using a temperature probe, as depicted in and described with reference to FIGS. 7A and 7B.

In operation 910, a treatment probe is inserted into an object. Operation 910 may be identical to operation 810, such that the treatment probe is inserted into an object through an exposed surface of the object or, alternatively, disposed outside of the object.

In operation 920, a desired temperature and duration are received. The desired temperature may be a desired temperature of a treatment volume. For example, computing device 130 may receive the desired temperature and/or duration from an operator via input/output element 120. The duration may be the desired duration at which the treatment volume is placed at the desired temperature, or may be the duration of an entire treatment (e.g., including heating and cooling times). The desired temperature and/or duration may be independently input for each of one or more treatment probes 150, where the desired temperature and/or duration may be the same for all treatment probes 150 or may be different for different probes 150.

In operation 930 acoustic waves are applied to a target volume. Operation 930 may be identical to operation 820. In some embodiments, the initial acoustic wave characteristics (e.g., intensity, pressure, etc.) may be determined based on the received desired temperature.

In operation 940, the temperature of the target volume is determined. Operation 940 may be identical to operation 830.

In operation 950, it is determined whether the temperature of the target volume is equal to the desired temperature. For example, controller 132 may compare the received desired temperature with the temperature of the target volume determined in operation 940. When the temperature of the target volume is equal to the desired temperature, processing may continue to operation 970. Otherwise, processing may continue to operation 960.

In operation 960, the acoustic waves being applied to the target volume are adjusted. Operation 960 may be identical to operation 840. Further, the acoustic waves may be adjusted based on whether the temperature of the target volume is greater than or less than the desired target volume temperature. For example, when the temperature of the target volume is greater than the desired target volume temperature, the acoustic waves may be adjusted to reduce the amount of energy absorbed by the target volume (e.g., by reducing the wave intensity, reducing the wave pressure, moving the wave direction of propagation away from the target volume, moving the wave focal depth away from the target volume, etc.). When the temperature of the target volume is less than the desired target volume temperature, the acoustic waves may be adjusted to increase the amount of energy absorbed by the target volume (e.g., by increasing the wave intensity, increasing the wave pressure, moving the wave direction of propagation toward the target volume, moving the wave focal depth toward the target volume, etc.).

In operation 970, it is determine whether the received duration is satisfied. For example, controller 132 may compare a duration over which the temperature of the target volume has been equal to the desired temperature with the desired duration received in operation 920 (in some embodiments, the difference could be within a range, such as between 0 and 0.5 degrees, between 0 and 1 degree, between 0.5 degrees and 2 degrees, or other suitable ranges). When the duration over which the temperature of the target volume has been equal to the desired temperature is less than the desired duration, processing may return to operation 930. Otherwise, the treatment process may end.

It should be appreciated that the specific operations illustrated in FIG. 9 provide a particular method of treating a target volume using acoustic waves, according to certain embodiments of the present invention. Other sequences of operations may also be performed according to alternative embodiments. Alternative embodiments of the present invention may also perform the operations outlined above in a different order. Moreover, the individual operations illustrated in FIG. 9 may include multiple sub-operations that may be performed in various sequences as appropriate to the individual operation. Furthermore, additional operations may be added or existing operations removed depending on the particular applications. This may include one or more of the variations described with reference to FIG. 8. For example, in some embodiments, treatment may include imaging the target volume. One of ordinary skill in the art would recognize and appreciate many variations, modifications, and alternatives.

### Additional Embodiments

While various embodiments are depicted and described with reference to in FIGS. 1 through 9, the scope of the disclosure is not so limited. In some embodiments, the treatment probes may not be limited to applying acoustic waves, but may be configured to generate and output waves at various different frequencies or various different heating energies. For example, one or more heating energy dispensing elements may be operable to apply acoustic waves, electromagnetic waves in one or more frequency bands, such as radio waves, microwaves, infrared waves, visible light waves, ultraviolet waves, laser, ionizing radiation, x-rays, gamma rays, etc. In some embodiments, one or more acoustic wave dispensing elements may be located external to the object, where precise real-time temperature measurement at each target volume is used as feedback to independently control the output wave characteristics of the external (or internal) acoustic wave dispensing elements. In some embodiments, the external acoustic wave dispensing elements may be spaced such that each target location is close enough to the adjacent space to minimize variability in tissue types throughout the target volume area. In some embodiments, one or more external acoustic wave dispensing elements may rotate a focus point such that it sweeps an entire target volume with multiple focus points in order to fully ablate and/or provide hyperthermia to a target volume. One of ordinary skill in the art would recognize and appreciate many variations, modifications, and alternatives.

In certain embodiments, systems, methods and devices as described herein have been demonstrated as remarkably effective in delivering energy to a target volume while more precisely controlling the resulting temperature applied to the target volume (e.g., controlled tissue heating). In accordance with various embodiments described herein, acoustic waves applied to target volumes can be specifically controlled, resulting in an unprecedented temperature control of target volumes in which treatment probes are disposed.

Target tissue heating involving systems, methods and devices described herein is not limited to any particular target temperature or temperature range. Delivery of heating energy as described herein, for example, may include heating of tissue from no discernable increase in tissue temperature above baseline (e.g., body temperature, such as normal human body temperature of about 37 degrees C.) to temperatures inducing indiscriminate, heat-mediated tissue destruction (e.g., tissue necrosis, protein cross-linking, etc.). For example, target tissue heating temperatures may include increases of target tissue from about 0 to about 5, 10, 20, 30 degrees C. (or higher) above baseline, as well as any temperature increment therebetween.

In some embodiments, heating energy application may be selected to elicit mild tissue heating, such that target tissue is heated a few degrees above baseline or body temperature, such as 0.1 to about 10 (or more) degrees Celsius above baseline or body temperature (e.g., 1, 2, 3, 4, 5, 6, 7, 8, 9, 10, 11, 12, etc. degrees Celsius above baseline). Such mild heating and/or accurate temperature control through a target volume can be particularly advantageous in applications where it is desired to destroy cancerous cells while minimizing damage to nearby healthy cells. For example, mild tissue heating may be selected such that wave delivery elicits preferential disruption or destruction to cancerous cells in a target tissue (e.g., target tissue volume) compared to non-cancerous cells in the target tissue.

As described above, systems, methods and devices described herein further allow for more precise control of the temperatures or temperature ranges of the target tissue or heating elicited in the target tissue with delivery of heating energy. Thus, target temperatures can include a target range or selected/expected deviation from the target temperatures. For example, tissue heating temperatures or ranges can include a modest deviation from a target, and will typically be less than a few degrees Celsius, and in some instances less than about 1 degree Celsius (e.g., 0.001 to about 1 degree Celsius). For example, actual heating may be from +/− about 0.001 to about 10 degrees Celsius, or any increment therebetween.

Throughout this description, reference may be made to various temperatures. Temperatures can be actual temperatures, predicted or calculated temperatures, or measured temperatures (e.g., directly or indirectly measured tissue temperatures). In some embodiments, such temperatures may correspond to the temperature of a treatment probe, subset of treatment probes, or all treatment probes disposed in a target volume. For example, treatment probe temperature may be acquired via a temperature sensor disposed in a treatment probe, such as temperature sensor 540 (FIG. 5A), but may also or alternatively be acquired via a temperature sensor disposed proximate the treatment probe or even outside of the target volume which the treatment probe are disposed in (e.g., via remote thermal sensing). Accordingly, in other embodiments, the temperatures may correspond not to the temperature of a treatment probe, but rather to the temperature of tissue or a target volume in contact with a treatment probe(s) or proximate a treatment probe(s). Further, the temperature may not be the actual temperature of the treatment probe or target volume, but rather, in some embodiments, could be an approximation or predicted temperature of the treatment probe or target volume. For example, the temperature of one treatment probe could be approximated by using a reading from a temperature sensor disposed in a proximate treatment probe. While not exact, the temperature of the proximate treatment probe may be a good approximation of the temperature of the treatment probe at issue as long as the treatment probes are disposed close enough to each other.

While embodiments of the present invention are described with particular reference to targeting tissue, systems, methods and devices described herein are not intended for limitation to any particular tissue or bodily location. For example, systems, methods and devices of the present invention can be utilized for targeting various different tissues including cancerous cells of various tissue types and locations in the body, including without limitation prostate, breast, liver, lung, colon, kidney, brain, uterine, ovarian, testicular, stomach, pancreas, etc.

Accordingly, the scope of the invention should be determined not with reference to the above description, but instead should be determined with reference to the pending claims along with their full scope or equivalents.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of delivering acoustic waves to a target tissue volume inside of a patient for medically treating the target tissue volume, the method comprising:
inserting a treatment probe into the patient through an exposed skin of the patient, the treatment probe including an acoustic wave dispensing element;
applying acoustic waves to the target tissue volume via the acoustic wave dispensing element, the acoustic waves being applied so as to medically treat the target tissue volume;
monitoring an amount of energy absorbed by the target tissue as a result of applying the acoustic waves; and
adjusting the acoustic waves being applied to the target tissue based on the amount of energy absorbed by the target tissue.

2. The method of claim 1, wherein the amount of energy is determined by measuring a temperature of the target tissue or by calculating an estimated temperature of the target tissue.

3. The method of claim 2, wherein the temperature of the target tissue is measured using one or more of a thermistor, a thermocouple, a magnetic resonance imager, an infrared temperature sensor, and an ultrasound temperature sensor.

4. The method of claim 2, wherein the temperature of the target tissue is estimated using one or more of the target tissue type, characteristics of the acoustic wave dispensing element, distance of the acoustic wave dispensing element from the target tissue; orientation of the acoustic wave dispensing element with respect to the target tissue, and characteristics of the applied acoustic waves.

5. The method of claim 1, wherein applying acoustic waves to the target tissue volume includes propagating acoustic waves to a lens disposed in the treatment probe.

6. The method of claim 1, wherein applying acoustic waves to the target tissue volume includes actuating an acoustic transducer disposed in the treatment probe.

7. The method of claim 1, further comprising:
inserting a plurality of treatment probes each having an acoustic wave dispensing element; and
applying acoustic waves to the target tissue volume via the acoustic wave dispensing elements.

8. The method of claim 1, wherein applying acoustic waves to the target tissue volume includes applying one or more of a transverse wave, focused wave, and a dispersed wave.

9. The method of claim 1, wherein the acoustic waves are applied to ablate at least some of the target tissue or induce hyperthermia in at least some of the target tissue.

10. The method of claim 1, further comprising imaging the target tissue volume using the acoustic wave dispensing element.

11. A system for delivering acoustic waves to a target tissue volume inside of a patient for medically treating the target tissue volume, the system comprising:
a treatment probe for treating the target tissue volume, the probe being insertable through an exposed skin of the patient and including an acoustic wave dispensing element operable to output acoustic waves for medically treating the target tissue volume;
a monitor operable to monitor an amount of energy absorbed by the target tissue as a result of applying the acoustic waves; and
a controller coupled to the wave dispensing element and the monitor, the controller operable to control the acoustic waves output by the wave dispensing element based on the amount of energy absorbed by the target tissue.

12. The system of claim 11, wherein the monitor calculates an estimated temperature of the target tissue.

13. The system of claim 11, wherein the monitor includes one or more of a thermistor, a thermocouple, a magnetic resonance imager, an infrared temperature sensor, and an ultrasound temperature sensor.

14. The system of claim 11, wherein the acoustic wave dispensing element includes one or more of a lens and an acoustic transducer.

15. The system of claim 11, further comprising a plurality of treatment probes each having an acoustic wave dispensing element, the probes being arranged to apply acoustic waves to the target tissue volume.

16. A treatment probe for delivering acoustic waves to a target tissue volume inside of a patient for medically treating the target tissue volume, the treatment probe comprising:
a housing having one end configured to pierce through exposed skin of the patient;
an acoustic wave dispensing element coupled to the housing and operable to output acoustic waves; and
a communication element coupled to the acoustic wave dispensing element and operable to communicate signals for controlling the acoustic waves.

17. The acoustic transducer of claim 16, further comprising a temperature sensor coupled to the housing and operable to measure a temperature of the target tissue volume.

18. The acoustic transducer of claim 16, wherein the acoustic wave dispensing element is an acoustic transducer, the communication element is a wire electrically coupled to the acoustic transducer, and the wire is operable to communicate control signals to the acoustic transducer for controlling the acoustic transducer to generate acoustic waves.

19. The acoustic transducer of claim 16, wherein the acoustic wave dispensing element is a lens, the communication element is a waveguide coupled to the acoustic transducer, and the waveguide is operable to communicate acoustic waves from a wave generator to the lens.

20. The acoustic transducer of claim 16, wherein the acoustic wave dispensing element is further operable to image the target tissue volume or measure a temperature of the target tissue volume.

21. A method of delivering heating energy to a target tissue volume inside of a patient for medically treating the target tissue volume, the method comprising:
inserting an array of treatment probes into the patient through an exposed skin of the patient, a plurality of the treatment probes each including a heating energy dispensing element;
applying heating energy to the target tissue volume via the dispensing elements, the heating energy being applied so as to medically treat the target tissue volume;
monitoring an amount of energy absorbed by the target tissue as a result of applying the heating energy; and
adjusting the heating energy being applied to the target tissue based on the amount of energy absorbed by the target tissue;
wherein the heating energy is selected from acoustic wave, microwave, infrared wave, visible light wave, ultraviolet wave, laser, or ionizing radiation energy.
